(19) United States
(12) Patent Application Publication
(10) Pub. No.: US 2023/0223157 A1
(43) Pub. Date: Jul. 13, 2023

(54) SYSTEM AND METHOD FOR DETECTING, FORESTALLING AND TREATING CANCER PATIENTS USING ARTIFICIAL INTELLIGENCE

(71) Applicant: KING KHALID UNIVERSITY, Abha (SA)

(72) Inventors: MOHAMMAD Y. ALSHAHRANI, Abha (SA); MUATH SULIMAN, Abha (SA); VINEET TIRTH, Abha (SA); VIBHA MALIK, Moradabad (IN); MANISHA BHATKULKAR, Wadi (IN)

(21) Appl. No.: 18/184,377

(22) Filed: Mar. 15, 2023

(51) Int. Cl. G16H 70/60; A61N 5/10; G06V 10/40; G16H 20/40; G06N 3/084

(52) U.S. Cl. CPC G16H 70/60; A61N 5/10; G06N 3/084; G06V 10/40; G16H 20/40

(57) ABSTRACT

The system comprises an input device for collecting a comprehensive pathological database obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions; a pre-processor for removing noise from collected comprehensive pathological database; a training processor configured with a deep learning technique for training back propagation network (BPN) using pathological tests of cancer patients and immunologically abnormal patients; a classification processor for categorizing patients based on extent of diseases using back propagation network (BPN); and a central processor equipped with a radiotherapy device for using the autoimmunity of the patients upon triggering the autoimmunity of the patients in the concerned tissue, organ or the blood for killing the cancer cells to control the infection.

### FIELD OF THE INVENTION

The present disclosure relates to a system and method for detecting, forestalling and treating cancer patients using artificial intelligence. In more detail, the system provides few strategies to target Histone acetyltransferases (HATs) or Adipose tissue macrophages (ATMs) to forestall cancer development or irritation, like macrophage consumption, bar of hostile to phagocytic flagging.

### BACKGROUND OF THE INVENTION

Macrophages have essential capabilities in homeostasis and numerous physiological cycles past natural resistance, including metabolic confiscation of dead cells, tissue redesigning, and safeguarding.

In pathogenic environments, TRMs is supplanted and amalgamated by selected monocyte-determined macrophages to coordinate a safe reaction. Current examinations utilizing single-cell RNA-sequencing (scRNA-seq) have uncovered the occurrence of numerous macrophage subgroups with unmistakable capabilities in different tissues.

For instance, in fatty liver, hepatic Kupffer cells (KCs) were obliterated and ousted by bone marrow-determined MPs, and those enlisted macrophages contain two subgroups looking like KCs or lipid-related MPs communicating osteopontin.

Also, macrophage activity and aggregation are affected by different elements in physiological and obsessive circumstances, like eating regimen and cytokines. The adjustment of macrophage aggregation or divergence is related to particular quality articulation profiles.

In the view of the forgoing discussion, it is clearly portrayed that there is a need to have a system and method for detecting, forestalling and treating cancer patients using artificial intelligence.

### SUMMARY OF THE INVENTION

The present disclosure seeks to provide a system and method for detecting, forestalling and treating cancer patients by triggering macrophages using artificial intelligence. “The Function of Macrophages in Disease analysis using Artificial Intelligence” includes Tissue-resident macrophages (TRMs), which are heterogeneous populaces starting from monocytes or undeveloped begetters, and convey in non-lymphoid and lymphoid tissues. TRMs participate in numerous physiological cycles, including metabolic activity, confiscating dead cells, tissue redesigning, and safeguarding. Macrophages (MPs) are one form of white blood cells, which play a significant role in neutralizing infectious microorganisms, stimulating immune cells, and removing dead cells. MPs is captivated by various useful aggregates relying upon their nucleating point and the microenvironment of the tissue. Explicit MPs accumulation happens in response to infection movement. Investigations on the sequencing of single-cell RNA strategies recognized a few basic particles to prompt the difference in MPs capability. These particles are possible markers for determination and particular focuses for novel macrophage-intervened treatment. This system presents discoveries concerning less-known particles and new elements of notable atoms. Understanding the components of these atoms in MPs is yield new macrophage-intervened medicines or symptomatic ways to deal with illness. MPs are a critical populace of inborn resistance, with strong effects on homeostasis, tissue fix, stoutness, and disease. MPs comprise two populaces, tissue-occupant cells with a pre-birth beginning and post-pregnancy monocyte-inferred ones. Independent of their starting point, MPs are exceptionally plastic, which can change their form as per the surrounding environment.

In an embodiment, a system for detecting, forestalling and treating cancer patients using artificial intelligence is disclosed. The system includes an input device for collecting a comprehensive pathological database obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions.

The system further includes a pre-processor for removing noise from the collected comprehensive pathological database.

The system further includes a training processor configured with a deep learning technique for training back propagation network (BPN) using pathological tests of cancer patients and immunologically abnormal patients.

The system further includes a classification processor for categorizing patients based on the extent of the diseases using the back propagation network (BPN), wherein the classification processor further forestalls or detects the cancer and cancer stage upon comparing the pathological test result of a new patient with the pathological database stored in a cloud server.

The system further includes a central processor equipped with a radiotherapy device for using the autoimmunity of the patients upon triggering the autoimmunity of the patients in the concerned tissue, organ or the blood for killing the cancer cells to control the infection.

In another embodiment, a method for detecting, forestalling and treating cancer patients using artificial intelligence is disclosed. The method includes collecting a comprehensive pathological database obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions via an input device.

The method further includes removing noise from the collected comprehensive pathological database through a pre-processor.

The method further includes training back propagation network (BPN) by a training processor using pathological tests of cancer patients and immunologically abnormal patients through a deep learning technique.

The method further includes categorizing patients based on the extent of the diseases using the back propagation network (BPN) using a classification processor, wherein the classification processor further forestalls or detects the cancer and cancer stage upon comparing the pathological test result of a new patient with the pathological database stored in a cloud server.

The method further includes using the autoimmunity of the patients upon triggering the autoimmunity of the patients in the concerned tissue, organ or the blood for killing the cancer cells to control the infection upon deploying a radiotherapy device through a central processor.

An object of the present disclosure is to provide Tissue-resident macrophages (TRMs), which are heterogeneous populaces starting from monocytes or undeveloped begetters, and convey in non-lymphoid and lymphoid tissues.

Another object of the present disclosure is to neutralize infectious microorganisms, stimulating immune cells, and removing dead cells.

Yet another object of the present invention is to deliver an expeditious and cost-effective system and method for detecting, forestalling and treating cancer patients.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for detecting, forestalling and treating cancer patients using artificial intelligence in accordance with an embodiment of the present disclosure;

FIG. 2 illustrates a flow chart of a method for detecting, forestalling and treating cancer patients using artificial intelligence in accordance with an embodiment of the present disclosure;

FIG. 3 illustrates the function of macrophages mode of action (MoA) in disease analysis using artificial intelligence flow in accordance with an embodiment of the present disclosure; and

FIG. 4 illustrates the function of macrophages in disease analysis using artificial intelligence structure in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises...a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

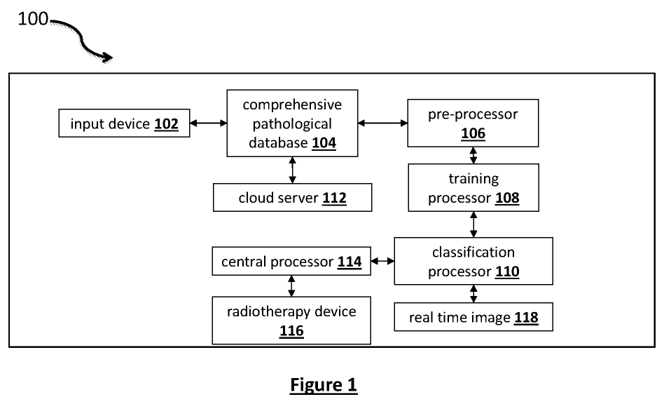

Referring to FIG. 1, a block diagram of a system for detecting, forestalling and treating cancer patients using artificial intelligence is illustrated in accordance with an embodiment of the present disclosure. The system 100 includes an input device 102 for collecting a comprehensive pathological database 104 obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions.

In an embodiment, a pre-processor 106 is used for removing noise from the collected comprehensive pathological database 104.

In an embodiment, a training processor 108 is configured with a deep learning technique for training back propagation network (BPN) using pathological tests of cancer patients and immunologically abnormal patients.

In an embodiment, a classification processor 110 is used for categorizing patients based on the extent of the diseases using the back propagation network (BPN), wherein the classification processor 110 further forestalls or detects the cancer and cancer stage upon comparing the pathological test result of a new patient with the pathological database stored in a cloud server 112.

In an embodiment, a central processor 114 is equipped with a radiotherapy device 116 for using the autoimmunity of the patients upon triggering the autoimmunity of the patients in the concerned tissue, organ or the blood for killing the cancer cells to control the infection.

In another embodiment, the five subsets emerge, three subsets based on cancer stages and red blood cells level in the patients and two stages from abnormal immune system, hypo and hyper, wherein the patients with hypo immune system are exposed to limit radiotherapy to trigger the growth of the cells hence, increasing the response of the immune system.

In another embodiment, the classification processor further may require a real time image of the suspicious area of the new patient to forestall or detect the cancer and cancer stage.

In another embodiment, the central processor 114 is further configured to target HAT or ATMs to forestall cancer development or irritation, like macrophage consumption, bar of hostile to phagocytic flagging including Siglec-10 or SIRPo, wherein understanding the atomic systems of macrophage capability in sickness gives strong symptomatic markers or potentially restorative methodologies by using artificial intelligence and translating cancer to autoimmunity.

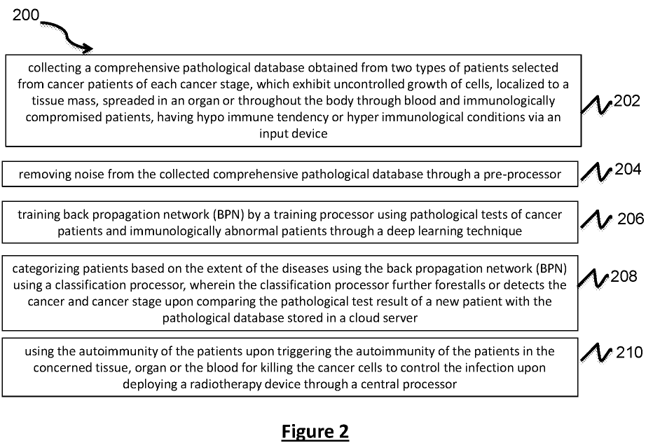

FIG. 2 illustrates a flow chart of a method for detecting, forestalling and treating cancer patients using artificial intelligence in accordance with an embodiment of the present disclosure. At step 202, the method 200 includes collecting a comprehensive pathological database 104 obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions via an input device.

At step 204, the method 200 includes removing noise from the collected comprehensive pathological database 104 through a pre-processor 106.

At step 206, the method 200 includes training back propagation network (BPN) by a training processor 108 using pathological tests of cancer patients and immunologically abnormal patients through a deep learning technique.

At step 208, the method 200 includes categorizing patients based on the extent of the diseases using the back propagation network (BPN) using a classification processor 110, wherein the classification processor 110 further forestalls or detects the cancer and cancer stage upon comparing the pathological test result of a new patient with the pathological database stored in a cloud server 112.

At step 210, the method 200 includes using the autoimmunity of the patients upon triggering the autoimmunity of the patients in the concerned tissue, organ or the blood for killing the cancer cells to control the infection upon deploying a radiotherapy device 116 through a central processor 114.

In another embodiment, the Tissue-resident macrophages (TRMs) participates in numerous physiological cycles, including metabolic activity, confiscating dead cells, tissue redesigning, and safeguarding, wherein Macrophages (MPs) is used for neutralizing infectious microorganisms, stimulating immune cells, and removing dead cells.

In another embodiment, forestalling and detecting the cancer comprises determining pathological test result and suspicious area image of a new patient. Then, converting suspicious area image into digital signal. Then, extracting image features using a feature extraction processor. Then, comparing the pathological test result and image features for categorizing patients based on the extent of the diseases to detect the cancer and its stage or forestalling the cancer and its stage if cancer is not detected.

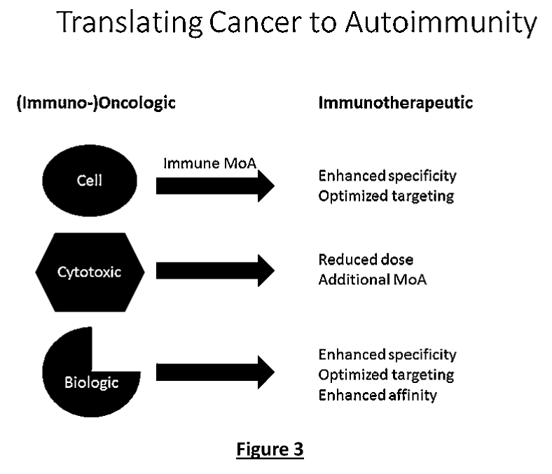

FIG. 3 illustrates the function of macrophages mode of action (MoA) in disease analysis using artificial intelligence flow in accordance with an embodiment of the present disclosure.

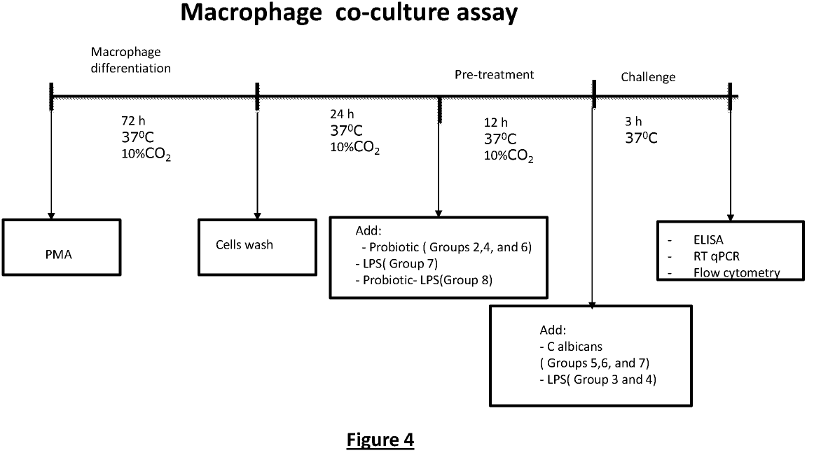

FIG. 4 illustrates the function of macrophages in disease analysis using artificial intelligence structure in accordance with an embodiment of the present disclosure.

Synovial tissue macrophages (STMs) assume basic parts in immune system sicknesses including rheumatoid artheritis (RA). With the examination of incorporated scRNA-seq, profound phenotypic, spatial and useful information is obtained. The STMs is extensively ordered into two populaces, MerTK–CD206– STMs and MerTK+CD206+ STMs. MerTK–CD206–STMs produce supportive of provocative cytokines, alarming and prompting fiery reactions in synovial fibroblasts, while MerTK+CD206+ STMs from patients with RA in supported illness reduce lipid production and initiate irritation.

This actuates aggregate of fibroblast-like synoviocytes. Additionally, two STM subpopulations (MerTK+TREM2high and MerTK+LYVE1+) with exceptional reduction of transcriptomic marks, causing poor control of irritation.

Additionally, a recognized subgroup of provocative MPs communicates heparin-restricting EGF-like development figure in RA affected joints, modifying synovial fibroblast quality articulation profile (e.g., IL-33) by epidermal development, factor receptor (EGFR) reaction, and expand their obtrusiveness.

MerTK being a key efferocytosis receptor, is communicated by CD11b+ F4/80+ huge peritoneal macrophages (LPMs). At consistent state, Mer–TK–lacking LPMs show altogether create favorable environment for incendiary cytokine articulation, clearing apoptotic cells, Mer-TK–/–LPMs expanded quality articulation of cell transitory and apoptosis.

They are typically enraptured into M1 or M2-like aggregate; nonetheless, ongoing examinations propose that the aggregate of macrophages can’t be essentially isolated into M1/M2 polarity, and extra characterizations, like Histone acetyltransferases (HATs) and Adipose tissue macrophages (ATMs), are applied to characterize the tissue-explicit MPs. This system features promising competitor macrophage because of their expected applications in control and treatment of illnesses.

A few strategies applied to target HAT or ATMs to forestall cancer development or irritation, like macrophage consumption, bar of hostile to phagocytic flagging (e.g., Siglec-10 or SIRPa). In this manner, understanding the atomic systems of macrophage capability in sickness gives strong symptomatic markers or potentially restorative methodologies by using artificial intelligence (deep learnng) and translating cancer to autoimmunity.

Deep learning technique based on back propagation (BPN) technique will work on a comprehensive pathological database 104 obtained from two types of patients; (1) Cancer patients of all stages (I, II, III), which exhibit uncontrolled growth of cells, localized to a tissue mass, spreaded in an organ or throughout the body through blood, and (2) Immunologically compromised patients, having hypo immune tendency or hyper immunological conditions (autoimmunity). The pathological tests of cancer patients and immunologically abnormal patients will be used to train the BPN network using deep learning and patients will be categorized based on the extent of the diseases. Five subsets will emerge, three subsets will be based on cancer stages and red blood cells level in the patients and two stages from abnormal immune system, hypo and hyper. The autoimmunity of the patients will be used by triggering the autoimmunity of the patients in the concerned tissue, organ or the blood to kill the cancer cells, hence controlling the infection. The patients with hypo immune system will be exposed to limit radiotherapy to trigger the growth of the cells hence, increasing the response of the immune system.

Presently, scRNA-seq is a basic instrument to examine the heterogeneity and capability of macrophage. For instance, five groups of alveolar macrophages (AMs) across homeostasis and intense irritation is revealed by transcriptional profiling examination.

In the course of settling aggravation, all communicating macrophage-explicit markers, such as CD68 and Lgals3 (Galectin-3), amid them, two bunches exhibit tissue-inhabitant territorial macrophage marker qualities, like Mrc1 (CD206) and Itgax (CD11c). Furthermore, three more bunches of critical upregulation of enrolled AM marker qualities, like CD14, Ly6c1 (Ly6c), and Sell (L-selectin) are recognized. Inhabitant AMs show higher articulation of M2-like MPs qualities and two groups of selected AMs have higher articulation of M1-like MPs qualities.

Though the last bunch of enlisted AMs display moderately low articulation of M1- M2 quality articulation profiles. This information further demonstrate that the M1-M2 worldview isn’t adequate for reasoning macrophage polarization. Hence this system features a few significant qualities of MPs capability and transcriptional profiling.

“The Function of Macrophages in Disease analysis using Artificial Intelligence” is a Tissue-resident macrophages (TRMs), which are heterogeneous populaces starting from monocytes or undeveloped begetters, and convey in non-lymphoid and lymphoid tissues.

The system reveals TRMs participation in numerous physiological cycles, including metabolic activity, confiscating dead cells, tissue redesigning, and safeguarding. Macrophages (MPs) are one form of white blood cells, which play a significant role in neutralizing infectious microorganisms, stimulating immune cells, and removing dead cells. MPs is captivated by various useful aggregates relying upon their nucleating point and the microenvironment of the tissue.

The system explains explicit MPs accumulation happens in response to infection movement. Investigations on the sequencing of single-cell RNA strategies recognized a few basic particles to prompt the difference in MPs capability.

The system reveals that macrophages are possible markers for determination and particular focuses for novel macrophage-intervened treatment. This system presents discoveries concerning less-known particles and new elements of notable atoms. Understanding the components of these atoms in MPs may yield new macrophage-intervened medicines or symptomatic ways to deal with illness.

The system reveals that macrophages are a critical populace of inborn resistance, with strong effects on homeostasis, tissue fix, stoutness, and disease. MPs comprise two populaces, tissue-occupant cells with a pre-birth beginning and post-pregnancy monocyte-inferred ones.

The system reveals that independent of their starting point, MPs are exceptionally plastic, which can change their form as per the surrounding environment.

The system concludes that the cancer translation to autoimmunity by triggering macrophages using deep learning technique of artificial intelligence is useful in analysis and control of diseases.

The functional units described in this specification have been labelled as devices. The functional units comprise input device 102, comprehensive pathological database 104, pre-processor 106, training processor 108, classification processor 110, cloud server 112, central processor 114, and radiotherapy device 116. A device is implemented in programmable hardware devices such as processors, digital signal processors, central processing units, field programmable gate arrays, programmable array logic, programmable logic devices, cloud processing systems, or the like. The devices are also be implemented in software for execution by various types of processors. An identified device is including executable code and may, for instance, comprise one or more physical or logical blocks of computer instructions, which may, for instance, be organized as an object, procedure, function, or other construct. Nevertheless, the executable of an identified device need not be physically located together, but may comprise disparate instructions stored in different locations which, when joined logically together, comprise the device and achieve the stated purpose of the device.

Indeed, an executable code of a device or module could be a single instruction, or many instructions, and may even be distributed over several different code segments, among different applications, and across several memory devices. Similarly, operational data is identified and illustrated herein within the device, and is embodied in any suitable form and organized within any suitable type of data structure. The operational data is collected as a single data set, or is distributed over different locations including over different storage devices, and may exist, at least partially, as electronic signals on a system or network.

In accordance with the exemplary embodiments, the disclosed computer programs or modules is executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs are written in exemplary programming languages that execute from memory on the device or from a hosted server, such as BASIC, COBOL, C, C++, Java, Pascal, or scripting languages such as JavaScript, Python, Ruby, PHP, Perl or other sufficient programming languages.

Some of the disclosed embodiments include or otherwise involve data transfer over a network, such as communicating various inputs or files over the network. The network may include, for example, one or more of the Internet, Wide Area Networks (WANs), Local Area Networks (LANs), analog or digital wired and wireless telephone networks (e.g., a PSTN, Integrated Services Digital Network (ISDN), a cellular network, and Digital Subscriber Line (xDSL)), radio, television, cable, satellite, and/or any other delivery or tunneling mechanism for carrying data. The network may include multiple networks or sub networks, each of which may include, for example, a wired or wireless data pathway. The network may include a circuit-switched voice network, a packet-switched data network, or any other network able to carry electronic communications. For example, the network may include networks based on the Internet protocol (IP) or asynchronous transfer mode (ATM), and may support voice using, for example, VoIP, Voice-over-ATM, or other comparable protocols used for voice data communications. In one implementation, the network includes a cellular telephone network configured to enable exchange of text or SMS messages.

Examples of the network include, but are not limited to, a personal area network (PAN), a storage area network (SAN), a home area network (HAN), a campus area network (CAN), a local area network (LAN), a wide area network (WAN), a metropolitan area network (MAN), a virtual private network (VPN), an enterprise private network (EPN), Internet, a global area network (GAN), and so forth.

The drawings and the forgoing description give examples of embodiments. Those skilled in the art will appreciate that one or more of the described elements may well be combined into a single functional element. Alternatively, certain elements is split into multiple functional elements. Elements from one embodiment is added to another embodiment. For example, orders of processes described herein is changed and are not limited to the manner described herein. Moreover, the actions of any flow diagram need not be implemented in the order shown; nor do all of the acts necessarily need to be performed. Also, those acts that are not dependent on other acts is performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A system for detecting, forestalling and treating cancer patients using artificial intelligence, the system comprises:
an input device for collecting a comprehensive pathological database obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions;
a pre-processor for removing noise from the collected comprehensive pathological database;
a training processor configured with a deep learning technique for training back propagation network (BPN) using pathological tests of cancer patients and immunologically abnormal patients;
a classification processor for categorizing patients based on the extent of the diseases using the back propagation network (BPN), wherein the classification processor further forestalls or detects the cancer and cancer stage upon comparing the pathological test result of a new patient with the pathological database stored in a cloud server; and
a central processor equipped with a radiotherapy device for using the autoimmunity of the patients upon triggering the autoimmunity of the patients in the concerned tissue, organ or the blood for killing the cancer cells to control the infection.

2. The system as claimed in claim 1, wherein the five subsets emerge, three subsets based on cancer stages and red blood cells level in the patients and two stages from abnormal immune system, hypo and hyper, wherein the patients with hypo immune system is exposed to limit radiotherapy to trigger the growth of the cells hence, increasing the response of the immune system.

3. The system as claimed in claim 1, wherein the classification processor further may require a real time image of the suspicious area of the new patient to forestall or detect the cancer and cancer stage.

4. The system as claimed in claim 1, wherein the central processor is further configured to target HAT or ATMs to forestall cancer development or irritation, like macrophage consumption, bar of hostile to phagocytic flagging including Siglec-10 or SIRPα, wherein understanding the atomic systems of macrophage capability in sickness gives strong symptomatic markers or potentially restorative methodologies by using artificial intelligence and translating cancer to autoimmunity.

5. A method for detecting, forestalling and treating cancer patients using artificial intelligence, the method comprises:
collecting a comprehensive pathological database obtained from two types of patients selected from cancer patients of each cancer stage, which exhibit uncontrolled growth of cells, localized to a tissue mass, spreaded in an organ or throughout the body through blood and immunologically compromised patients, having hypo immune tendency or hyper immunological conditions via an input device;
removing noise from the collected comprehensive pathological database through a pre-processor;
training back propagation network (BPN) by a training processor using pathological tests of cancer patients and immunologically abnormal patients through a deep learning technique;
categorizing patients based on the extent of the diseases using the back propagation network (BPN) using a classification processor, wherein the classification processor further forestalls or detects the cancer and cancer stage upon comparing the pathological test result of a new patient with the pathological database stored in a cloud server; and
using the autoimmunity of the patients upon triggering the autoimmunity of the patients in the concerned tissue, organ or the blood for killing the cancer cells to control the infection upon deploying a radiotherapy device through a central processor.

6. The method as claimed in claim 5, wherein the Tissue-resident macrophages (TRMs) participates in numerous physiological cycles, including metabolic activity, confiscating dead cells, tissue redesigning, and safeguarding, wherein Macrophages (MPs) is used for neutralizing infectious microorganisms, stimulating immune cells, and removing dead cells.

7. The method as claimed in claim 5, wherein forestalling and detecting the cancer comprises:
determining pathological test result and suspicious area image of a new patient;
converting suspicious area image into digital signal;
extracting image features using a feature extraction processor; and
comparing the pathological test result and image features for categorizing patients based on the extent of the diseases to detect the cancer and its stage or forestalling the cancer and its stage if cancer is not detected.
